(19) United States
(12) Patent Application Publication
(10) Pub. No.: US 2023/0082699 A1
(43) Pub. Date: Mar. 16, 2023

(54) DEVICES WITH THIRD PARTY CONNECTIVITY

(71) Applicant: Ably Medical AS, Alesund (NO)

(72) Inventor: Arve Voldsund, Leinoy (NO)

(21) Appl. No.: 17/799,463

(22) PCT Filed: Feb. 12, 2021
(86) PCT No.: PCT/EP2021/053564

§ 371 Date: Aug. 12, 2022

(30) Foreign Application Priority Data
Feb. 12, 2020 (GB) 2001929.5

(51) Int. Cl. G16H 40/63

(52) U.S. Cl. CPC G16H 40/63

(57) ABSTRACT

The present disclosure relates to systems and methods that relate to the transfer of data between a person support structure (e.g., a hospital bed or chair) and third party software, in particular systems and methods for simplifying how a third party application uses data from the person support structure hospital bed and the exchange of data between a control system of the bed (e.g., local to the a bed or hospital) and a third party application (e.g., separated remote from the bed or hospital, such as via a network).

### FIELD OF THE INVENTION

The present invention relates generally to systems and methods for transferring data between a person support structure (e.g., a bed or chair), or monitoring device associated with such a structure, and third party software. The systems and methods are aimed at simplifying how a third party application uses data from the support structure (or monitoring device) and the exchange of data between a control system and a third party application remote therefrom, such as via a network.

### BACKGROUND

Patient/person support structures, such as hospital beds are becoming increasingly complex and new types of support structures are being developed that can quickly and in real-time determine certain patient features. Various types of monitoring devices are associated with such structures, including traditional bedside monitoring equipment as well as more complex devices that can be placed on or within the support structure.

Support structures have been developed, for example, that use sensors connected to resilient members that run along the length of the bed, from which various types of information can be ascertained. This information can relate to the tension or change thereof in the resilient members, which can be monitored over time by a control system in order to track, e.g., movement of the patient.

Recent developments in support structures and monitoring devices thereof have improved types of non-urgent monitoring of a patient. For example, a patient's physiological condition can be monitored using various devices for measuring the mechanical activity of a patient's body. Such monitoring can now generate a wide variety of data relating to the patient and/or device, in some cases from multiple devices and/or different types of sensors associated with the bed.

The present invention aims to make better use of the wide variety of data generated by the monitoring system(s) of a person support structure (and/or sensor devices within such structures), such as a hospital bed, in particular with third party application software and the accessibility of this data with such software.

### SUMMARY

In accordance with an aspect of the invention, there is provided a system for exchanging data between one or more structures for supporting a person (or “support structures”) and third party application software. The system comprises:

the one or more support structures, wherein each support structure (e.g., a device within the structure) is configured to output data corresponding to a person resting on the support structure;

one or more control systems configured to receive the data from the support structure(s);

one or more third party service modules, each being configured to store and execute third party application software that uses the data from the support structure(s);

a service request module for communicating data between the control system(s) and the one or more third party service modules; and

a data transfer module for communicating data between the control system(s) and the one or more third party service modules,

wherein the service request module is configured to receive a service request and communicate this request to a respective one of the third party service modules, wherein the service request corresponds to a request to provide analysis of the data from the support structure(s) using third party application software contained in the respective third party service module,

wherein the one or more third party service modules are each configured to analyse a service request received from the service request module, determine the data required to provide the analysis of the data from the support structure(s) using (and/or to execute) the respective third party application software and communicate a data request to the service request module, wherein the data request corresponds to a request for the data required to provide the analysis of the data from the support structure(s) using (and/or to execute) the respective third party application software,

wherein the service request module is configured to receive the data request, and then pass this to the control system(s) so as to notify the control system(s) of the data required to provide the analysis of the data from the support structure(s) using the third party application software,

wherein the one or more control system(s) are then configured to set up a data connection with the respective third party service module via the data transfer module, wherein the one or more control system(s) are configured to transfer to the third party service module, via the data transfer module, the data required to provide the analysis of the data from the support structure(s) using the third party application software,

wherein the third party service module is then configured to execute the third party application software so as to provide the analysis of the data from the support structure(s).

The same principles may be applied to any type of patient monitoring device. As such, from an aspect (which may be claimed independently) the invention provides a system for exchanging data between one or more devices and third party application software. The system comprises:

the one or more devices, wherein each device is configured to monitor a person and to output data corresponding to that person;

a control system in data communication with the devices and configured to receive the data output therefrom;

one or more third party service modules, each being configured to store and execute third party application software that uses the data from the device(s);

a service request module for communicating data between the control system and the one or more third party service modules; and

a data transfer module for communicating data between the control system and the one or more third party service modules,

wherein the service request module is configured to receive a service request and communicate this request to a respective one of the third party service modules, wherein the service request corresponds to a request to provide analysis of the data from a respective one of the devices using third party application software contained in the respective third party service module,

wherein the one or more third party service modules are each configured to analyse a service request received from the service request module, determine the data required to provide the analysis of the data from the respective device using the third party application software and communicate a data request to the service request module, wherein the data request corresponds to a request for the data required to provide the analysis of the data from the respective device using the third party application software,

wherein the service request module is configured to receive the data request, and then pass this to the control system, so as to notify the control system of the data required to provide the analysis of the data from the respective device using the third party application software,

wherein the control system is then configured to set up a data connection with the respective third party service module via the data transfer module, wherein the control system is configured to transfer to the respective third party service module, via the data transfer module, the data required to provide the analysis of the data from the respective device using the third party application software,

wherein the respective third party service module is then configured to execute the third party application software so as to provide the analysis of the data from the respective device.

In accordance with an aspect of the invention, there is provided a method for exchanging data with third party application software. The method may be used with either of the above systems.

The method may comprise providing the one or more support structures or monitoring devices.

The method may comprise providing the control system.

The method may comprise providing the one or more third party service modules.

The method may comprise providing the service request module.

The method may comprise providing the data transfer module.

The method may comprise generating the service request.

The method may comprise the service request module receiving the service request and communicating this request to the respective third party service module.

The method may comprise the respective third party service module analysing the service request received from the service request module, determining the data required to provide the analysis of the data from the support structure(s) or device(s) using the third party application software and communicating a data request to the service request module, wherein the data request corresponds to a request for the data required to provide the analysis of the data from the support structure(s) or device(s) using the third party application software.

The method may comprise the service request module receiving the data request, and then passing/transmitting this to the control system so as to notify the control system of the data required to provide the analysis of the data from the support structure(s) or device(s) using the third party application software.

The method may comprise the control system then setting up a data connection with the respective third party service module via the data transfer module, wherein the control system then transfers to the third party service module, via the data transfer module, the data required to provide the analysis of the data from the support structure(s) or device(s) using the third party application software.

The method may comprise the third party service module executing the third party application software so as to provide the analysis of the data from the support structure(s) or device(s).

In one sequence, the method may comprise:

using the service request module to receive a service request and then communicate the service request to a respective one of the third party service modules;

using the respective third party service module to analyse the service request, determine the data required to provide the analysis of the data from the respective device using the third party application software, and then communicate a data request to the service request module;

using the service request module to receive the data request, and then to transmit this to the control system so as to notify the control system of the data required to provide the analysis of the data from the respective device using the third party application software;

setting up a data connection between the control system and the respective third party service module via the data transfer module; and

transferring the data required to provide the analysis from the control system to the third party service module, via the data transfer module.

The above described systems (and methods) provide improvements in how data generated from a support structure or monitoring device can be accessed by third party application software. Using the various modules in the manner described above means that the third party application does not have to be downloaded to the support structure or device, and can be accessed more easily by a user.

Non-medical applications are contemplated, and references to “patient” and “caregiver” herein are not intended to limit the embodiments to medical applications, and the terms “patient” and “caregiver” are interchangeable with any terms that refer to a person that might lie on, use or operate the method and/or control system as appropriate, for example “user”, “controller” or “operator”.

The following features relate to optional features of the present invention, including any of the aspects of the invention described above.

In aspects involving a support structure, this may be a bed or other structure (e.g., a chair such as a wheelchair) configured to support a person (e.g., at rest). The support structure may include hardware configured to control a movement of the structure to aid in supporting the person resting thereon (such as a moving portion of a chair/bed).

The support structure may contain hardware for diagnostic, therapeutic and/or surgical means to be applied to the person resting thereon. Any of this hardware may be configured to output the data corresponding to the person resting thereon, for example using sensors or other data producing means.

Each support structure may comprise one or more devices for monitoring a person resting thereon (“sensor device” or “monitoring device”), for example the device may comprise one or more sensors configured to measure characteristics of the person resting thereon (e.g., movement sensors, as discussed in more detail below) and output data via sensory outputs. The device(s) may be configured to be placed within the support structure, such as above or below a mattress or cushion thereof.

In any of the aspects described herein, the data communicated between the control system and the one or more third party service modules via the service request module (e.g., first data) may be different to the data communicated between the control system and the one or more third party service modules via the data transfer module (e.g., second data). For example, the service request (e.g., first data) may be a data packet containing information including, for example, details of the analysis to be provided and/or the third party service module able to provide such analysis. The data request (e.g., second data) may be a data packet containing information including, for example, details of the data required to provide the analysis requested in the service request, such as identification of certain sensory outputs of the support structure to be monitored to collect the data, as well as (for example) information for assisting such monitoring, such as a monitoring time and/or frequency.

The systems and methods described herein may be implemented across local and/or wide area networks.

A local area network may be within a single physical building or site (e.g., care facility), and/or could transfer data using an Ethernet or WiFi connection. A wide area network may correspond to transfer of data via the Internet and/or mobile telephone network (e.g., using a satellite, 4G, 5G or other similar connection).

For example, the system may comprise a local area network, wherein the service request module, the data transfer module, and (optionally) one or more of the third party service modules, may be implemented using software on the local area network. Additionally or alternatively, one or more of the third party service modules could communicate with the other modules via a wide area network (which is in communication with the local area network). This provides a useful way of interconnecting the various modules of the system, in that the data transferred between them can be limited as much as possible to a local area network. The local area network may be located within a building or facility (e.g., a hospital or other type of care facility).

The support structures or monitoring devices of the system may be interconnected with the various modules (which can be on the same local area network) via the local and/or a wide area network. For example, one device could be located in a patient home and communicate with the modules via a wide area network (e.g., the internet), another device could be located in an ambulance and communicate with the modules via a wide area network (e.g., mobile telecommunication network), and a plurality of devices could be located within the same care facility (e.g., hospital) and communicate with the modules via the same local area network.

The service request module and data transfer module may be embodied on the same computing device, such as a server, or as part of a cloud-based program or ‘virtual server’. In either case the service request module and data transfer module may be located on a single local area network (e.g., of a single care facility).

Moving now to the data output from the support structure(s) or device(s), this may correspond to physiological data, such as one or more of body temperature, blood pressure, pulse (heart rate), and breathing rate (respiratory rate). The data may correspond to body position or posture, or movement data.

The system may comprise one or more sensors located on, within or associated with (e.g., connected to) each support structure or monitoring device. For example, one or more of temperature sensors, blood pressure sensors, pulse (heart rate) sensors, breathing rate (respiratory rate) sensors, and other vital sign sensors. The sensors may collect these and other types of health data corresponding to the person.

An advantage of this system is that is allows selection of data from various different sensory outputs to be used as part of a bespoke third party analysis. Accordingly, the analysis of the data may comprise a diagnosis relating to the person, and the diagnosis may include an analysis of various different data sources (from different sensors), such as an analysis of different types of data to provide a higher level analysis (e.g., a health assessment based on multiple data types/sources).

The systems described herein may be particularly effective for providing third party analysis based on the position, posture or movement of a person.

The system may comprise one or more sensors configured to output position, posture or movement data, which sensors may include piezoelectric sensors and/or any other suitable sensors.

It has been found that position, posture or movement data may be particularly useful when combined with the systems and methods described herein. Such details may be useful in various types of diagnosis or other analysis, and may change frequently over time. As such, the disclosed technology allows various different types of third party application software to be used to analyse such movement, and without having to download all of the various different software applications to the support structure or monitoring device, or update such equipment constantly with the changes in such software applications over time.

In an optimised arrangement of movement sensors, the one or more sensors may comprise at least 2, 3, 4 or 5 movement sensors arranged in an array (e.g., a parallel array). The movement sensors could be configured (e.g., when placed in a support structure) to be located adjacent (e.g., beneath) the torso and/or buttocks of a person. For example, the movement sensors may be attached to resilient members that provide support (e.g., the primary support) for the person at least in the torso and/or buttocks region.

Moving now to the control system, this may be implemented on one or more computing devices in data communication with each of the support structures or monitoring devices. The control system may be embodied on the same computing device, such as a server, or as part of a cloud-based program or ‘virtual server’ (which could be the same as that referred to above in respect of the service request and data transfer modules). In any case the control system may be located on a single local area network (e.g., a single care facility). Providing the control system on a centralised server (physical or virtual) can simplify the communication between the various modules and the support structures or monitoring devices.

In any of the aspects described above, the system may further comprise a user interface configured to display a plurality of analysis options, each being associated with a respective third party service module, and to allow a user to select one or more of the analysis options, wherein such selection lead to the generation of one or more service requests to provide analysis of the data using third party application software contained in the respective third party service module(s).

These embodiments permit a plurality of different third party services to be used, as well as a simple method of choosing such services and allowing transfer of data to the third party service modules. These embodiments additionally reduce the amount of data processing, as well as meaning that the devices and control system can be simplified (e.g., kept free of third party software).

Regarding the service request module, this may be configured to determine if the data identified in the data request by the third party service module is accurate and/or appropriate and/or required (e.g., for the analysis at hand), and approve the data request for further transmission to the control system if it is determined that the data identified in the data request is accurate and/or appropriate and/or required (e.g., for the analysis at hand).

These embodiments provide a mechanism for checking whether the data requested by the third party service module is appropriate before approving the data request and forwarding it on to the control system. “Appropriate” in this sense could mean that the service request module checks the type of data requested (e.g., pulse rate) against the service requested (e.g., analysis of heart condition) and confirms that the type of data is appropriate (e.g., required) for the analysis at hand. The service request module could include a database including types of data that are appropriate/required for each analysis available so that it can perform this assessment. Alternatively, a user (e.g., caregiver such as a doctor) could be presented with the type of data requested and be prompted to confirm (e.g., using a user interface such as those described herein) themselves that it is appropriate/required for the analysis at hand.

Regarding the data transfer module, this may be configured to verify that the data being transferred between the control system and the third party service module corresponds only to data identified in the data request, and forward the data from the control system to the third party service module only if this is the case. If not the case, the data transfer module may be configured to output an error notification (e.g., message).

These embodiments provide a mechanism for checking/confirming that the data transferred to the third party service module, i.e., from the control system, corresponds only to the data identified in the request, which as discussed elsewhere herein prevents inadvertent misuse of data. As the data to be transferred could correspond to private health data of a person on the support structure, this is seen as an important refinement of the present invention.

Regarding the third party service module, this may be configured to send the analysis back to the control system via the data transfer module. The analysis, e.g., analytical information, could be in any suitable form, such as a results table or other data including the outcome of the analysis or the analytical information. Once received by the control system the analysis could be displayed on a user interface (e.g., as described herein).

The third party application software may be configured, when run, to analyse data received via the data transfer module. Such data may correspond to sensory data that has been output from sensors on the support structure or monitoring device, which sensor data may be raw sensor data, or sensor data that has been processed before being passed to the data transfer module. The processing may be averaging or sampling, or more complex such as a determination of one or more of body temperature, blood pressure, pulse (heart rate), and breathing rate (respiratory rate).

The only data that is transferred to the third party service module may be the service request, data request, data from the support structure(s) or monitoring device(s) and analysis (e.g., analytical data). This limits the data use of the systems and methods described herein and the exposure of the third party to such data.

In some embodiments the data transfer module may be configured to log the amount and/or type of data transferred between the control system and the third party service module, and output a record of the amount and/or type of data that was required to provide the analysis. These embodiments allow additional monitoring of the data transferred within the system, wherein the record can provide useful information regarding the use of such data.

Unless otherwise indicated, the various method steps, functional elements, stages, “modules” and “means” of the invention (e.g., the system described above, including the third party service modules, service request module, data transfer module, broker module and any other module) may comprise a suitable processor or processors, controller or controllers, functional units, circuitry, processing logic, microprocessor arrangements, etc., that are operable to perform the various functions, etc., such as appropriately dedicated hardware elements and/or programmable hardware elements that can be programmed to operate in the desired manner.

The various method steps, functional elements, stages, “modules” and “means” of any aspects or embodiments of the present invention (e.g., the system described above, including the third party service modules, service request module, data transfer module, broker module and any other module) may be implemented at least partially using software, e.g., computer programs.

For example, references to “application” and/or “software” could be interpreted as being implemented at least partially using software, e.g., computer programs. It will thus be seen that when viewed from further aspects the present invention provides computer software specifically adapted to carry out the method steps, functional elements, stages, “modules” and “means”, etc., herein described (e.g., the system described above, including the third party service modules, service request module, data transfer module, broker module and any other module) when installed on data processing means, a computer program element comprising computer software code portions for performing the methods herein described when the program element is run on data processing means, and a computer program comprising code means adapted to perform all the steps of a method or of the methods herein described when the program is run on a data processing system. The data processor may be a microprocessor system, a programmable FPGA (field programmable gate array), etc.

The present invention may accordingly suitably be embodied as a computer program product for use with a computer system. Such an implementation may comprise a series of computer readable instructions either fixed on a tangible, non-transitory medium, such as a computer readable medium, for example, diskette, CD ROM, ROM, RAM, flash memory, or hard disk. It could also comprise a series of computer readable instructions transmittable to a computer system, via a modem or other interface device, over either a tangible medium, including but not limited to optical or analogue communications lines, or intangibly using wireless techniques, including but not limited to microwave, infrared or other transmission techniques. The series of computer readable instructions embodies all or part of the method steps and/or functional features described herein.

The software or computer system adapted to carry out the method steps, functional elements, stages, “modules” and “means” of any aspects or embodiments of the present invention (e.g., the system described above, including the third party service modules, service request module, data transfer module, broker module and any other module) may be implemented on a single device, multiple devices (for example devices connected by a local or wide area network, such as a “cloud”-type network).

### BRIEF DESCRIPTION OF THE DRAWINGS

Various embodiments will now be described, by way of example only, and with reference to the accompanying drawings in which:

FIG. 1A shows an embodiment of a support structure that may be used with the various systems and methods disclosed herein;

FIG. 1B shows the support structure of FIG. 1A having moved from the configuration shown in FIG. 1A to a different configuration;

FIG. 2 shows schematically a system according to the present invention that includes a plurality of support structures connected to third party application software via one or more intermediate components and/or modules; and

FIG. 3 shows a flow chart illustrating the system described in respect of FIG. 2, but in terms of users/devices/applications, as opposed to modules.

### DETAILED DESCRIPTION

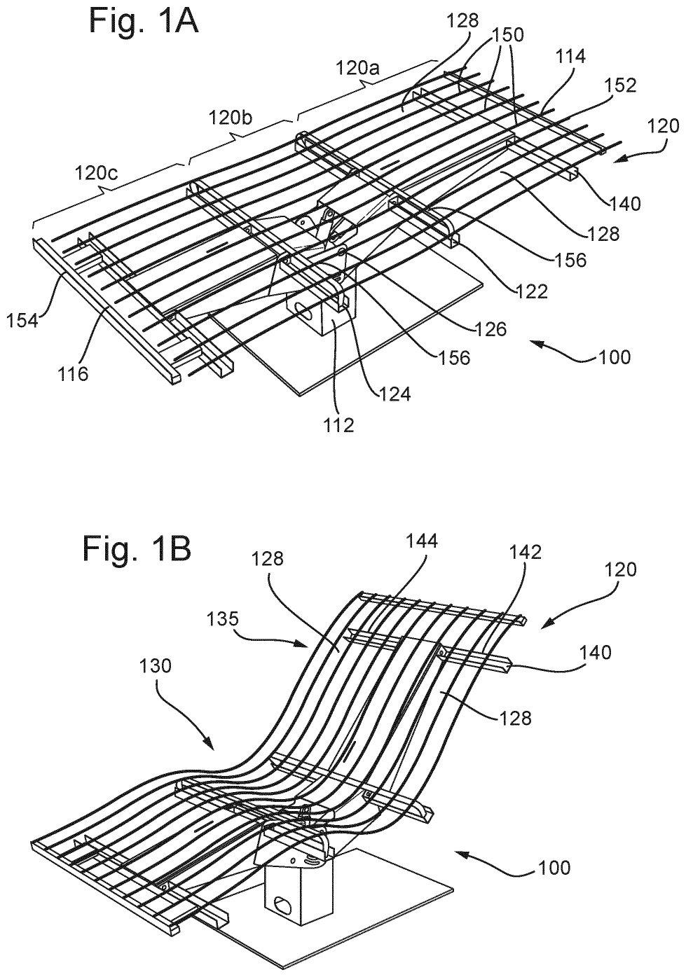

A support structure 120 will now be described with reference to FIGS. 1A-1B. In the illustrated embodiment the support structure 120 is shown as forming part of a bed 100.

The present invention is not limited to the support structure shown and described, and the skilled person will appreciate that various other types of structure (e.g., a chair) may be used, as well as other types of bed. For example, although a support structure with a plurality of moving sections is described, this is not essential and a static or simpler structure could be used. It will also be appreciated that certain aspects of the present invention relate to the use of a monitoring device configured to monitor a person and to output data corresponding to that person, rather than specifically a support structure (although the monitoring device could be part of a support structure or connected thereto).

The illustrated support structure 120 comprises a plurality of sections 120a, 120b, 120c each configured to support a respective part of a person's body. In the illustrated embodiment of FIG. 1A a first section 120a is configured to support a person's upper body, including the back and head, a second section 120b is configured to support a person's upper legs and buttocks, and a third section 120c is configured to support a person's lower legs.

Although three sections are shown in FIG. 1A, any suitable number of sections may be provided, for example only two sections, or more than three.

The first section 120a may have a length of between about 0.7-1.1 m, optionally about 0.8 m. The second section 120b may have a length of between about 0.4-0.5 m, optionally about 0.45 m. The third section 120c may have a length of between about 0.5-0.6 m, optionally about 0.55 m. The length may correspond to the lengthwise or longest dimension of the support structure 120. The support structure 120 may extend along the entire length of the bed 100.

Each section may be separated from an adjacent section by at least one transverse separation line (e.g., a pivot point) 122, 124.

In the illustrated embodiment, a first separation line 122 may separate the first section 120a from the second section 120b, and a second separation line 124 may separate the second section 120b from the third section 120c. The first and second separation lines 122, 124 may correspond to the major points of flexure of a human, as discussed above, namely the knees and waist. A crossbeam or lateral support bar may be located at each of the first and second separation lines 122, 124.

A central pivot point 126 may be located at approximately the centre of the bed 100, for example at the point at which a bed support 112 meets the support structure 120, such that the support structure 120 of the bed 100 can rotate as a whole about the central pivot point 126. The central pivot point 126 may not necessarily be located at a point of flexure, and/or may be located at a point between the first and second separation lines 122, 124.

The support structure 120 may have a length equal to or greater than about 1.5 m, about 1.6 m, about 1.7 m, about 1.8 m, about 1.9 m, about 2.0 m, about 2.1 m, about 2.2 m or about 2.3 m. The length may correspond to the lengthwise or longest dimension of the support structure. The support structure 120 may extend along the entire length of the bed 100.

The support structure 120 may have a width equal to or greater than about 0.8 m, 0.9 m, about 1 m or about 1.1 m. The width may correspond to a direction perpendicular or transverse to the length.

The support structure 120 may be raised from the ground by a height of between about 0.1-1 m, about 0.2-0.9 m, about 0.3-0.9 m, or about 0.5-0.9 m.

The support structure 120 may comprise a plurality of sensor elements configured to provide an electrical response as a function of (e.g., proportional to) the amount of movement of a person resting therein.

The support structure 120 may comprise resilient members 150 to support a person thereon, which may be an elongate member (for example a spring, such as a band spring), wherein the one or more sensor elements may extend along at least some (or substantially all) of the length of the member to allow measurement of changes in movement due to changes in one or more of pressure, acceleration, strain, or force associated with the resilient member.

The resilient members 150 may each comprise a sensor element that is attached to, and/or integrated into, and runs at least partially along the length of the respective resilient member 150.

The sensor elements may be provided in any suitable configuration. It is not essential, for example, that they are attached to resilient members 150 as described.

Each of the sensor elements is configured to provide an electrical response as a function of (e.g., proportional to) the amount of movement of the person, for example by detecting movement of a respective resilient member. For example, the sensor elements may be configured to generate an electrical charge, current or voltage resulting from a mechanical force applied to them (e.g., applied to the respective resilient member 150). The charge, current or voltage may be proportional to the amount of mechanical force applied to the resilient member 150.

The plurality of resilient members 150, for example, carbon, organic or glass fiber composite members (e.g., springs) may extend in the longitudinal (i.e., lengthwise or longest) direction from an upper end 114 of the support structure 120 to a lower end 116 of the support structure 120. The resilient members 150 may be held in place at (e.g., attached to) the upper end 114 by an upper bracket 152, and at the lower end 116 by a lower bracket 154. For example, the resilient members 150 may be attached or connected to (e.g., slotted or hooked into) the upper and lower brackets 152, 154, for example using suitable locking members.

The upper and/or lower bracket 152, 154 may comprise crossbeams or lateral support bars to which the resilient members 150 are attached or connected.

The resilient members 150 may be supported by or otherwise attached to further brackets 156 at each separation line. For example, the resilient members 150 may be attached or connected (e.g., slotted into complimentary grooves) to the further brackets 156, for example to the crossbeams or lateral support bars that are located there (if provided). The further brackets 156 may be configured to hold the resilient members 150 against lateral (side-to-side) movement, which provides a convenient way of holding the resilient members 150 in a parallel array as described elsewhere herein.

The resilient members 150 may be configured to support a person lying on the support structure 120 and/or may provide the primary support for a person. While it is envisaged that a further material (e.g., a mattress, foam or memory foam, which is not shown in FIGS. 1A-1B) may be provided above (or itself encase) the resilient members 150, the shape and/or profile of the support structure 120 may be determined by (e.g., correspond to) the shape and/or profile of the resilient members 150, as shown in more detail in FIG. 1B. The shape and/or profile of the resilient members 150 may also correspond to the shape and/or profile of the sensor elements, for example due to the sensor elements being connected continuously along the length of a respective resilient member 150.

The various sections of the support structure 120 may be independently movable (e.g., up and down) and/or rotatable about their respective separation lines 122, 124. As the various sections of the support structure 120 rotate the resilient members 150 may be configured to change shape. In other words, the resilient members 150 may be biased so as to form a predefined shape and/or profile upon rotation of the various sections of the support structure 120. The shape and/or profile of the resilient members 150 (and therefore the support structure 120) may be different in each section.

As shown in the illustrated embodiment of FIG. 1B, upon rotation of the first section 120a about the first separation line 122, and/or the third section 120c about the second separation line 124, the resilient members 150 may be configured to form a convex profile in the first section 120a and/or the third section 120c respectively, and may be configured to form a concave profile in the second section 120b.

References to “concave” and “convex” as used herein should be interpreted as being towards a person (e.g., patient) lying on the bed and in the longitudinal direction, for example such that a concave profile forms a depressed portion (e.g., a dip or valley) of the bed in a longitudinal direction, and a convex profile forms a raised portion (e.g., a bump or protrusion) of the bed in a longitudinal direction.

For example, upon rotation of the sections 120a and 120b from a flat position (as shown in FIG. 1A) into a more upright position (as shown in FIG. 1B) the support structure 120 automatically provides a concave profile 130 (e.g., a pit or valley) as well as a convex profile 135 for lumbar support. The use of longitudinal resilient members 150 (as opposed to vertical springs or lateral members) allows such profiling to be more easily tailored for an intended use of the bed.

The resilient members 150 may be configured, in the flat and upright positions of the support structure 120, to substantially conform to the shape of the body. For example, when the support structure 120 is in a flat position the resilient members 150 may preferably undulate to follow the contour of a body in a lying down position, or less preferably the resilient members 150 may be flat. When the support structure 120 is in an upright position, the support structure 120 may undulate to follow the contour of a body in a seated position. It will be appreciated that the undulations in the resilient members 150 when the support structure 120 is in the seated position may be more pronounced than the undulations in the resilient members 150 when the support structure 120 is in the flat position.

There may be no lateral resilient members or springs provided in the support structure 120. The resilient members 150 may have a length equal to or greater than about 1.5 m, about 1.6 m, about 1.7 m, about 1.8 m, about 1.9 m, about 2.0 m, about 2.1 m, about 2.2 m or about 2.3 m. The length may correspond to the lengthwise or longest dimension of the support structure.

The support structure 120 may comprise at least 5, 6, 7, 8, 9, 10, 15 or 20 sensor elements (and/or resilient members 150, e.g., in a parallel array) and/or the sensor elements (e.g., resilient members) may be spaced apart by less than 5, 10, 15 or 20 cm, to provide sufficient sensory response or (in the case of the resilient members) support to a person on the support structure 120.

One or more sensor elements may be connected to, and/or integrated/embedded into, one or more (or all of) the resilient members 150, which sensors may be configured to provide an electrical response as a function of (e.g., proportional to) the amount of movement of the respective resilient member as described above.

The sensor elements may be a piezoelectric material (e.g., cable), for example that runs along the length of each respective resilient member 150, and configured to measure the piezoelectric (i.e., electrical) response caused by movement of a person resting on the support structure (e.g., movement of the resilient members 150).

It will be appreciated that a piezoelectric response of such sensor elements will be at a minimum (or zero) when there is no movement (e.g., in the resilient members 150), and will increase upon increased movement. For example, there may be a very high tension in the resilient members 150, but the piezoelectric response may still be at a minimum (or zero). Thus, the use of piezoelectric sensor elements is seen as a particularly advantageous development over merely measuring, e.g., tension, since it gives an improved response to and/or more information regarding the movement of a person being supported on the support structure 120. This is particularly the case when the sensor elements are attached to (e.g., embedded in or encased by) the resilient members forming the primary support for the support structure, since they will respond directly to movement of the resilient members. This is in contrast to conventional arrangements that incorporate a pad within a mattress for example, but do not attach the sensor elements to the resilient members of the mattress that provide primary support (e.g., the mattress springs).

More generally, the use of sensor elements that run along at least part of the length of the support structure (e.g., resilient members) leads to a desirable trade-off, as discussed above. That is, the sensor elements disclosed herein can respond to changes in movement, e.g., a person moving from one side to another, or having a seizure/sneezing/coughing, etc., as well as smaller movements such as breathing, heart rate fluctuations, abdomen noises and so forth.

The sensor elements also permit fast and simple detection of sudden changes in movement, which in the case of a bed may be caused by a person as they are about to fall off the bed (for example). The use of a resilient member 150 as disclosed herein (i.e., comprising a sensor element running through or along it) means that a caregiver response (or a response that uses the movement of the support structure 120) can be faster.

The movement in each sensor element could be monitored by a system configured to measure the electrical (e.g., piezoelectric) response from the sensor elements.

The system could be capable of moving the portions of the bed, such that an automatic response to the sensor elements can be provided. For example, if it becomes apparent that the person is moving towards the side of the bed, for example due to the various movements of the resilient members 150 that are spaced laterally across the bed, the system may determine that the person is about to fall off the bed, and take corrective action. In the illustrated example, the combination of resilient members 150, and their spacing laterally across the support structure 120 (e.g., as shown in FIGS. 1A-1B) is seen as particularly advantageous when combined with such a system.

The system may be configured to sound an alarm or otherwise alert a caregiver (or other person) prior to the person actually falling off the bed. In embodiments where the bed comprises one or more movable portions, the system may move (e.g., raise) a suitable portion of the bed in order to prevent the person falling off.

The movement in the sensor elements (and, e.g., resilient members 150) could be monitored over time. Based on the change in the movement in the sensor elements over time the system may determine movement patterns of the person, some of which may lead to an alert. For example, if the electrical (e.g., piezoelectric) response from the sensor elements is substantially stable, and/or follows a normal pattern then the system may determine that the person is stable and/or moving normally and continue monitoring. If the movement becomes unstable, and/or follows an abnormal pattern (e.g., due to the person thrashing or writhing) then the system could sound an alarm or otherwise alert a caregiver (or other person).

In some embodiments the sensor elements (and, e.g., resilient members) may extend along the majority of, or the entire length of the support structure 120. In other embodiments the sensor elements may extend partially along the length of the support structure 120, for example the portion of the support structure 120 corresponding to the torso and/or buttocks of a person. Providing the sensor elements at least in the portion of the support structure 120 corresponding to the torso and buttocks means that most of the important movement characteristics can be measured whilst limiting the material required for the sensor elements. Alternatively, for a complete/optimum arrangement the sensor elements could extend the entire length of the support structure.

Various parts of the support structure 120 may be movable or rotatable in order to provide further automated movement possibilities for a person, in addition to the rotation about the first and/or second separation lines 122, 124, and/or the central pivot point 126.

For example, the upper corners 128 of the support structure 120 may be adjustable such that they can be raised or lowered independently of each other and/or the other parts of the support structure 120. This can provide a movement configured to lift the shoulder of a person lying on the bed.

To effectuate such movement a support bar 140 may be located at or near the upper end 114 of the support structure 120. The support bar 140 may comprise a left arm 142 and a right arm 144, both of which may be independently raised or lowered. One or more motors (not shown) may be provided to raise and lower each of the left arm 142 and right arm 144.

A similar arrangement may be placed at the lower end 116 of the support structure 120 in order to raise and lower the legs or feet of a person lying on the bed.

Other movements are envisaged (but not essential). The support structure 120 may be configured such that it can be raised and/or lowered about a longitudinal axis of rotation, for example the central longitudinal axis of the support structure 120. For example, each separation line may comprise a support bar similar to the support bar 140, wherein the support bars may be configured to simultaneously raise all of the right or left arms, so that one half of the support structure 120 is raised. Such a movement may assist in turning a person.

In various embodiments, the support structure 120 may comprise a plurality of sections (e.g., at least two or three sections, e.g., corresponding to any number (or all) of the sections 120a, 120b, 120c), and all or part of the sections may be movable by a translating means, e.g., other than rotation about a pivot point. For example, each section may be movable (e.g., up and down) independently of the other sections. Additionally, or alternatively, a portion of each section may be movable (e.g., up and down) independently of the rest of the section, and/or independently of the other sections.

The portion of each section may be independently movable by configuring the resilient members 150 such that each resilient member 150 is independently movable within that portion of the section. For example, suitable actuators could be provided for the resilient members 150 that may be configured to move the resilient members 150 up and down within a particular section, or within a portion of a particular section. In some embodiments separate actuators could be provided for each resilient member 150 that are configured to move each resilient member separately within a particular section, or within a portion of a particular section.

In accordance with the invention, there is provided various systems and/or methods for exchanging data with a third party application that is executed remotely from a support structure or monitoring device.

The systems and methods described herein may be implemented at least partially on a local area network, e.g., at a single healthcare facility, although it is envisaged that the implementation could include communication via a wide area network (e.g., from the healthcare facility to an ambulance, or a person's home, etc. The various modules may be implemented on a ‘virtual server’ on existing hardware (e.g., server(s)/central processing system(s)) at the facility.

Some of the support structures or monitoring devices could be located at the same facility and on the same local area network (e.g., a number of hospital beds), whilst others could be located in a separate geographical location (e.g., in an ambulance or at different homes). The modules described herein could connect to each other via the local and wide area networks as required.

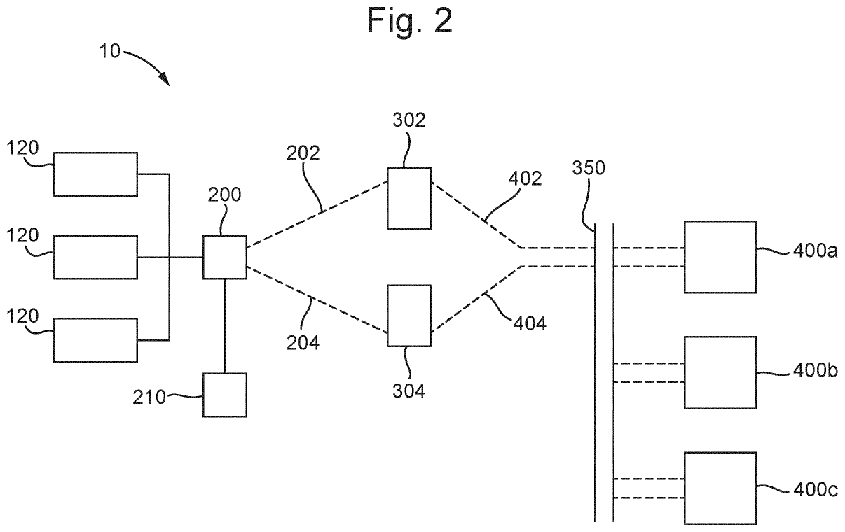

FIG. 2 shows schematically a system 10 including a plurality of support structures 120, which may each correspond to a support structure 120 as described above.

As noted above the system 10 is not limited to use with the specific support structure 120 described above and any support structure (e.g., chair, bed, etc.) could be used. The support structures could be the same or different. In addition, aspects of the invention relate to the use of devices configured to monitor a person (as opposed to specifically support structures). Therefore, the references to “support structures 120” in the following description could also refer to monitoring devices.

The system 10 is for enabling connectivity between the support structures 120 (or monitoring devices) and a third party service or application located remote therefrom.

The system 10 further comprises a control system 200 configured to communicate with each of the support structures 120, as well as receive data therefrom, such as patient data, diagnostic data and other types of data.

The control system 200 may be provided by a single computing device (e.g., server), or as part of a cloud-based ‘virtual’ server. In either case the control system 200 may be located on a local area network connected to each of the support structures 120 and configured to communicate with each support structure 120.

The control system 200 may be configured to provide a third party service. The types of services envisaged may include diagnostics, therapies, and monitoring capabilities for a person using the support structure (e.g., a patient in a care facility, such as a hospital). The services also extend to monitoring the support structure itself, such as services that monitor the condition or other characteristics of the support structure.

The control system 200 may be configured to communicate with each support structure 120 or monitoring device via a wired or wireless communications link.

The control system 200 may have a user interface 210 to enable a patient or caregiver to provide diagnostics, therapies and monitoring capabilities for the person. The user interface 210 may comprise one or a plurality of input devices, for example touchscreen devices, that enable a user (e.g., patient or caregiver) to select a third party service, such as a certain type of diagnostic process, therapy or monitoring capability. The user interface 210 may list the types of third party services or applications that are available, and permit a user to select (or request) a service to be used.

It is envisaged that the user interface 210 could be embodied on one or more mobile computing devices such as tablet computers, personal digital assistants, smart phones or other such devices. This devices may be configured to “pair” with the control system 200 such that data or other information associated with the support structures 120 can be accessed or otherwise visible on the user interface 210.

It is further envisaged that the user interface 210 could be embodied on a web browser or other web application. This could communicate via a wired or wireless communications link, for example as part of a local area network of the care setting or facility.

The third party services may be services that use sensory data obtained from the support structure(s) 120 or monitoring device(s).

The third party services could relate to a diagnostic process that uses data obtained from the support structure(s) 120 or monitoring device(s), such as respiratory data from a respiratory monitor, and/or movement data.

In accordance with the invention, the processing of such data to provide the third party analysis is carried out remotely from the support structure or monitoring device. The third party service and/or application may be located on the local area network of the hospital, but access by the third party application software to the support structure or monitoring device may be restricted.

In some embodiments the third party service or application could be run at a location remote from the support structures (such as remote from the building, care facility or a local area network thereof). Communication between the third party service or application and the rest of the system could be via a wide area network (e.g., the internet).

Where more than one third party service or application is provided, some of them could be run locally to the care facility (e.g., on a local area network), and others could be run at a remote location (e.g., via a wide area network).

The present invention aims to simplify how a third party application uses the data in order to provide its service.

In contrast to some conventional arrangements the present invention does not aim to provide third parties with the ability to run applications (e.g., software) on the support structure(s) 120. Instead, the technology and invention disclosed herein is aimed at avoiding this, by controlling how data produced by the support structures 120 or monitoring devices is used, and simplifying the use of such data with third party applications. For example, it is desired to improve how the data output from the support structure(s) 120 (or monitoring devices) can be provided to third parties, so that they can more efficiently implement services using third party software, but without needing to authorise or otherwise use/run the third party application software locally.

For these purposes the system 10 further comprises a service request module indicated schematically at 302, and a data transfer module 304. Both the service request module 302 and the data transfer module 304 communicate with the control system 200 via respective data links 202, 204. The term “module” as used herein follows the definition given above in terms of implementation.

It is envisaged that third parties wishing to use the data output from the support structure(s) 120 or monitoring devices cannot access such data unless authorised to do so by the service request module 302. It is further envisaged that the data transfer module 304 can be used to limit the access of the third party to the data being output from the support structure.

The system 10 further comprises one or more third party service modules 400a-c, each being configured to store and execute third party application software (at least one software application) that uses data receivable from a support structure 120 or monitoring devices.

The third party service modules 400a-c may be located on the same local area network (and, e.g., at the same care facility) as the service request module 302 and the data transfer module 304. The third party service modules 400a-c could additionally (or alternatively) be located at a different geographical location, such as a home, ambulance, etc.

As used herein, a “service request” may correspond to a request to provide analysis of the data output from one or more of the support structures or monitoring devices using third party application software.

The service request module 302 and the data transfer module 304 are both configured to communicate with the third party service modules 400a-c via respective data links 402, 404. In various embodiments, the data links 402, 404 may be provided over a local area network (e.g., the same network as the support structures 120 or monitoring devices), or a wide area network (e.g., the internet). The latter would allow a third party to make their application available to users in different geographical locations.

The service request module 302 and data transfer module 304 may be embodied on the same computing device, such as a server, or as part of a cloud-based program or ‘virtual server’. Data transmitted through the modules 302, 304 may be controlled and/or monitored by an intermediary (e.g., intermediate application/software) between the control system 200 and the third party service modules.

The purpose of the service request module 302 and data transfer module 304 is to provide an interface for controlling the transfer of data between the control system 200 and the third party service modules 400a-c, such that data from one or more of the support structures 120 or monitoring devices can be exchanged therebetween.

As will be discussed in more detail below, following a request for a particular third party service, the modules 302, 304 can provide the third party application with the data required to fulfill their service. The outcome of the analysis may then be sent to the person or device requesting the service.

Generally, therefore, a support structure 120 or monitoring device may be configured to output data corresponding to the health of a patient. A proprietary third party application could be used to analyse such data and provide information based on the data, for example that could assist in care for the patient. It may not be in the interests of either a care facility, or the third party that the application/software itself is passed from the third party to the support structure(s) 120 or monitoring device. As such, a caregiver could request, e.g., via the user interface 210 as a “service request”, that the third party provides a service without having to take control of, install or otherwise use the third party application locally.

The control system 200 may be configured to pass a service request via the data link 202 to the service request module 302, which may be configured to analyse the request, carry out any analysis thereof, and then if appropriate forward the service request to the appropriate third party service module (e.g., module 400a) via the data link 402, which could be configured to analyse the request and determine the data required to satisfy it.

The third party service module 400a may then be configured to notify the service request module 302 of the data required to satisfy the request via the data link 402 (“data request”). The service request module 302 may be configured to verify/approve the data required by the third party service module 400a, and then (if approved) inform the control system 200 (via the data link 202) that the request for data has been approved, as well as notify the control system 200 of the data required to satisfy the request.

Examples of the data request 513 include a request for a time averaged pulse rate, which may involve a sensory output (pulse) processed (e.g., by the control system or data transfer module) to a time averaged pulse rate, which is then sent in a data packet 516 to the third party service provider 502.

The control system 200 may then set up a connection with the third party service module 400a via the data transfer module 304, wherein the control system 200 may be configured, via the connection, to transfer to the third party the data required to satisfy the service request.

The data transfer module 304 may be configured to check that the data being sent or forwarded to the third party service module 400a contains only the data required to satisfy the service request, and transfer the data only if this is the case. This can ensure that the third party (e.g., the third party application/software) is not provided with unnecessary data or given access to confidential information.

In this manner, the data may be transferred from the support structure 120 or monitoring device to the third party service module 400a, via the data transfer module 304 and the respective data links 204, 404.

The third party service module 400a may then be configured to execute the third party application so as to provide a suitable response, for example results, diagnosis or other analysis. This may involve analysing the data to provide the service that was originally selected by a user, for example results, diagnosis or other analysis based on the data provided to the third party.

The third party service module 400a may then be configured to send the response (e.g., results or diagnosis) back via the data transfer module 304 and data links 404, 204 for the benefit of the person or device that requested the service.

As will be appreciated this process enables a third party to provide a service remotely. It is envisaged that any third party could benefit from the processes described herein to the extent that they can use data that is output from a support structure or monitoring device.

The only data/information that is traded between the support structure 120 and the third party may be the service request, data request, and patient data specific to the service that was requested. As discussed above this limits the processing of unnecessary data and helps to prevent access to confidential information.

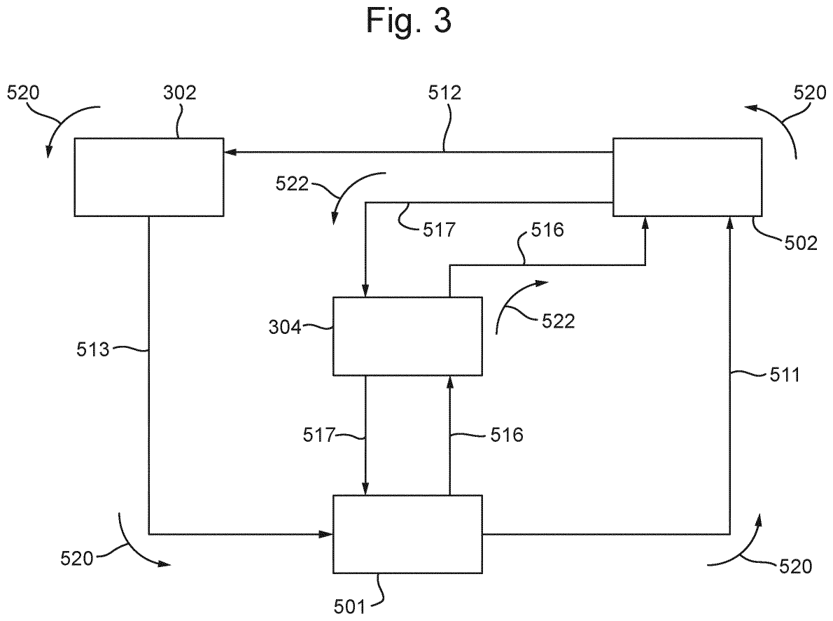

FIG. 3 shows a flow chart illustrating the above process in terms of users/devices/applications, as opposed to modules (although it represents substantially the same process).

A service requester 501, which may be a person, device or application software (e.g., part of control system 200), sends a service request 511 (e.g., via a user interface and/or control system 200, and the service request module 302) to a third party service provider 502, which may be a person, device or application software (e.g., part of a third party service module 400a-c).

The third party service provider 502 (e.g., the respective service module 400a-c thereof) then analyses and responds to the service request 511 by sending a data request 512 to the service request module 302, which then is configured to approve (or disapprove) the request for data.

This approval/disapproval may be an automated process built into the service request module 302, e.g., as part of application software thereof. The approval/disapproval may additionally (or alternatively) incorporate a prompt to a user of the service request module 302 to approve or disapprove the data request 512 from the third party service provider 502. This prompt may be by way of the user interface.

If approved, the service request module 302 forwards the approved data request 513 back to the service requester 501, which notifies the service requester 501 of the data required to provide the service as well as notification of the approval. The mere receiving of the forwarded data request by the service requester 501 could constitute an implicit approval, and as such the data request 513 received by the service requester 501 could be substantially identical to the data request 512 sent by the third party service provider 502.

This process so far may be seen as a first loop, or service loop (indicated by arrows 520) between the service requester 501, third party service provider 502 and the service request module 302, wherein the service loop is configured to identify the third party service required, as well as the data required to satisfy it and communicate this to the relevant parties.

Once the service loop is complete, a data exchange can be set up, which begins with the service requester 501 receiving the approved data request 513 and then setting up a data connection between the service requester 501 and the third party service module 400a, via the data transfer module 304.

To do this, the service requester 501 may send one or more data packets 516 of the required data, as identified in the data request, to the data transfer module 304, which then is configured to forward the data packets 516 (or a modified/processed version thereof) to the third party service provider 502.

The service requester 501 may be different to the device that produces the data packets 516, in which case the service requester 501 may obtain the required data and relay this to the data transfer module 304. In various embodiments the data packets 516 may be sent directly to the data transfer module 304, for example without being relayed through the service requester 501.

As noted above the third party service provider 502 (e.g., the third party service module 400a-c thereof) may then be configured to execute a third party application or software that analyses the data and provides a suitable response 517, for example results, diagnosis or other analysis. The response 517 may be communicated back to the data transfer module 304.

In various embodiments or situations the third party service provider 501 (e.g., the third party service module 400a-c thereof) may provide a response 517 in the form of a request for additional data, as opposed to results or a diagnosis, which may be the result of analysing the data packets 516 that were sent and determining that additional data is required in order to satisfy the service that was requested.

The data connection between the third party service provider 501 and the service requester 501 may be a streaming connection, wherein data to be streamed across the data connection is restricted (e.g., by the data transfer module 304) to only the data required to satisfy the service request.

The data transfer module 304 may be configured to receive the response 517 from the third party service provider 501 and may then be configured to forward the response 517 (or a modified/processed version thereof) and/or a notification to the service requester 501 to notify them that the service has been satisfied.

In the case of a request for additional data, the data transfer module 304 may forward the response 517 as a request for additional data to be processed by the service requester 501 or device. Should a request for additional data be received by the service requester 501 or device, then this will be processed in the same manner as the data request 513 received from the service request module 302.

The data exchange may be seen as a second loop, or data loop (indicated by arrows 522) between the service requester 501 and the data transfer module 304 and third party service provider 502, wherein the data loop is configured to transfer the required data between the parties, as well as the results of any analysis carried out at the third party service provider 501.

The data loop may be executed or initiated only after adequate completion of the service loop. In other words, data exchange may only take place upon approval of the third party service by the service request module 302. The data transfer module 304 may be configured to check that the data packet 516 being sent or forwarded to the third party service provider 501 only contains data identified in the approved data request 513, and only forward the data packet 516 (or modified/processed version thereof) if this is the case. As noted above this can ensure that the third party is not provided with unnecessary data or given access to confidential information.

The system may comprise a broker module configured to identify appropriate third party service modules 400a-c based on a particular service request.

The broker module may be used to simplify the selection of an appropriate third party service, or a number of services. For example, various care plans could be listed as one of a number of care plans on the user interface, each care plan being associated with a subset of third party services. The caregiver could then select the desired care plan without having to specify (or even be aware of) the third party services.

The broker module could then automatically choose the appropriate third party services to be used in connection with the care plan, and instruct the service request module 302 to communicate a plurality of service requests 511 for forwarding on to respective third party service providers 502 as aforesaid.

As described in more detail below the system 10 may comprise a module configured to monitor the data packets 516, 517 sent through the data transfer module, for example track the amount of data in the data packets 516, 517, or the number of packets of data sent, and the like. In this manner the system 10 can keep track of all data transactions.

In its broadest aspects, the technology described herein (e.g., using the above service and data loops) provides an effective way of managing the processing of data by third party applications.

For example, the above described processes enable the use of a wide (even infinite) variety of third party applications, without having to provide the third party (e.g., a third party application/software) with undesired access to data.

The technology described herein also enables the simplification of the support structures 120 or monitoring devices, since these parts of the system 10 do not have to be concerned with whether they can (or cannot) run third party applications. Rather, the only requirement is that the relevant data can be forwarded by the control system 200 to the third party as described above.

It is further envisaged that third party service providers could send proposals for third party services to a service requester, for example at a care facility, such that the service requester (e.g., care provider) could select from a plurality of potential services one that is most suitable for a given patient. The information that the third party service provider would need to do this may be an outline of the data that can be provided by the support structures 120 (or monitoring devices).

In accordance with aspects of the invention, a transaction method has been developed for use in combination with the methods described above.

The transaction method in various embodiments can enable monetisation of the methods described herein, e.g., a “pay per use” type of arrangement between the third party service provider 502, and either the service requester 501 and/or an intermediary in control of the service request module 302 and the data transfer module 304. The transaction method is not limited to a “pay per use” type of arrangement, and in its broadest aspects permits monitoring the transactions to allow any type of monetisation, or other type of monitoring that is desired.

The intermediary may be an application/software provided, for example, by a manufacturer of the support structure(s) or monitoring device(s).

In various embodiments, and as noted above, the transaction method may not be “pay per use”, but would still be useful in that the data transferred through the data transfer module 304 can be logged, for example to track the amount of data, type of data, etc. used by the third party in providing the service.

In order to keep track of the transactions, each data packet 516, 517 may be provided with a marker or other form of identification, which may be unique for each data packet 516, 517. Any analytical information provided by the third party may also be provided with a marker or other identification (which may also be unique). The system, for example one or more of the modules thereof, may be configured to match up the data packets 516, 517 with the service request and/or the analysis provided by the third party, so as to monitor and follow the transactions in a manner that permits investigation, billing and the like.

The monitoring of the transactions could be used to provide information, for example via a user interface, wherein the information could be presented in any format. In one example, the information regarding transactions could be provided as a graph and/or table, with the data packets 516, 517 and any associated analyses being shown, so that this information is readily available for future lookup or review.

The intermediary may be a manufacturer of the support structures 120 or monitoring devices used (e.g.) within a particular care facility, wherein such an intermediary would have knowledge of the types of data that could be determined therefrom.

For example, the third party service provider 502 may propose one or more services to the service requester 501, for example via the control system 200 and/or user interface 210. This could be a list of possible services that somehow make use of the data that can be determined from the support structures 120 or monitoring devices, such as patient data. At this point, the possible services could be provided with a price list associated with each service, or other type of pricing structure (e.g., based on an amount or number of data packets, or amount of data generally).

The service requester 501 can then select from the list a given service, which initiates the methods and processes described above in respect of FIGS. 2 and 3.

During this process, an intermediary in control of the service request module 302 and data transfer module 304 could monitor the service and data requests being transferred through the modules 302, 304. In doing so, they could monitor the amount and type of data that is being transferred within the data packets sent between the service requester 501 and the third party service provider 502.

In various embodiments, the intermediary may initiate a charge to the third party service provider 502, which may be based on the type of service being requested, and/or the data being sent within the data packets. It may then be up to the service provider 502 to choose whether to pass on the costs to the service requester 501 (e.g., care facility). The passage of data through the data transfer module 304 could be made conditional, for example on satisfaction of payment or otherwise by the third party service provider 502 to the intermediary.

In other embodiments the intermediary may initiate a charge to the service requester 501 (e.g., care facility) that may similarly be based on the type of service being requested, and/or the data being sent. As discussed above various pricing structures are possible. It would again be up to the third party service provider 502 to choose whether to pass on the costs of the service to the service requester 501. The passage of data through the data transfer module 304 could be made conditional, for example on satisfaction of payment or otherwise by the service requester 501 to the intermediary.

In other embodiments it is envisaged that the third party service provider 502 could initiate a charge to the intermediary, who could then choose whether to pass on the costs to the service requester 501 (e.g., care facility). The passage of data through the data transfer module 304 could be made conditional, for example on satisfaction of payment or otherwise by the service requester 501.

In accordance with any of the aspects and embodiments described herein, it is envisaged that any suitable type of data could be used for being sent to the third party. For example, and as discussed above, the support structures or monitoring devices may comprise any number of sensors that are configured to determine various types of data that relate to person/patient characteristics.

One representative example would be a support structure that comprises a respiratory monitor that is configured to collect data relating to the breathing characteristics of a patient in a care facility. A third party may have developed (or be able to develop) proprietary application software that can analyse the data from the respiratory monitor and determine one or more outcomes, such as a diagnosis or other analytical result. However, the third party may not want to disclose the details of this software, or go to the effort of providing it on a device developed by a different manufacturer.

The processes described herein permit the third party service provider to propose a service to the service requester (e.g., within the care facility), without having to necessarily go through the intricate details of the application or software itself. The service requester can then decide whether to request the service, and potentially outlay the cost for the same, whilst the third party is safe in the knowledge that their application software will not be disclosed.

Upon deciding that the service from the third party is suitable for the care situation at hand, the service requester may then send the service request as detailed above in the service loop 520, which initiates the process of obtaining the service from the third party. Throughout this entire process, no details of the third party application or software needs to be disclosed outside of the service provider 502 (e.g., third party service module 400a-c thereof). The service provider 502 can simply receive the data, process this accordingly and then send the results back to the service requester 501 as required.

An intermediary could be in control of the service request module 302 and the data transfer module 304, as noted above. They could then track the data being transferred between the parties (namely the service requester 501 and provider 502), and monitor the transactions to ensure that the relevant data and results are being properly transferred.

The processes described herein allow the service request module 302 and the data transfer module 304 to analyse or process the service request and data request, for example the data transfer module 304 may modify or process the data packets 516 so that the data is structured or formatted in a way that the third party application or software can analyse it and send their results.

The service request module 302 and data transfer module 304 could be located on a virtual server, e.g., located on or forming part of a local area network at the care facility, such as using “cloud” type application software.

An aim of the present invention is to reduce the requirement of care facilities to run different types of third party applications on multiple, different support structures or monitoring devices. Through the use of the processes described herein a third party application does not have to be downloaded to, or run locally. The processes described herein also benefit the third party, since they allow simplification of the use of data by their third party applications. For example, they may not have to disclose details of their software or applications. Rather, all that is traded between the two parties can be data packets containing either data from the support structure(s) or monitoring device(s), and the results of the service (e.g., the results of the third party application software).

Although the present invention has been described with reference to preferred embodiments, it will be understood by those skilled in the art that various changes in form and detail may be made without departing from the scope of the invention as set forth in the accompanying claims.

What is claimed is:

1. A system for exchanging data between one or more devices and third party application software, the system comprising:
the one or more devices, wherein each device is configured to monitor a person and to output data corresponding to that person;
a control system in data communication with the devices and configured to receive the data output therefrom;
one or more third party service modules, each being configured to store and execute third party application software that uses the data from the device(s);
a service request module for communicating data between the control system and the one or more third party service modules; and
a data transfer module for communicating data between the control system and the one or more third party service modules,
wherein the service request module is configured to receive a service request and communicate this request to a respective one of the third party service modules, wherein the service request corresponds to a request to provide analysis of the data from a respective one of the devices using third party application software contained in the respective third party service module,
wherein the one or more third party service modules are each configured to analyze a service request received from the service request module, determine the data required to provide the analysis of the data from the respective device using the third party application software and communicate a data request to the service request module, wherein the data request corresponds to a request for the data required to provide the analysis of the data from the respective device using the third party application software,
wherein the service request module is configured to receive the data request, and then pass this to the control system, so as to notify the control system of the data required to provide the analysis of the data from the respective device using the third party application software,
wherein the control system is then configured to set up a data connection with the respective third party service module via the data transfer module, wherein the control system is configured to transfer to the respective third party service module, via the data transfer module, the data required to provide the analysis of the data from the respective device using the third party application software,
wherein the respective third party service module is then configured to execute the third party application software so as to provide the analysis of the data from the respective device.

2. The system as claimed in claim 1, wherein the data corresponding to the person comprises data relating to one or more of posture, position and movement of the person.

3. The system as claimed in claim 1, wherein the data corresponding to the person comprises data relating to one or more of body temperature, blood pressure, pulse (heart rate), and breathing rate (respiratory rate).

4. The system as claimed in claim 1, wherein the analysis of the data comprises a diagnosis relating to the person.

5. The system as claimed in claim 1, further comprising one or more structures for supporting the monitored person, wherein each device is associated with (e.g., located on or within) a respective support structure.

6. The system as claimed in claim 5, wherein the support structure is a bed or chair.

7. The system as claimed in claim 1, wherein each support structure comprises one or more sensors configured to detect movement of a person resting on the support structure, wherein the data corresponding to the person comprises data relating to movement of the person that is received from the one or more sensors.

8. The system as claimed in claim 7, wherein the one or more sensors comprise at least five sensors arranged in an array across the support structure.

9. The system as claimed in claim 1, wherein the control system is connected by a wired or wireless connection to each monitoring device, such that data output from the monitoring devices is transferred to the control system via the wired or wireless connection.

10. The system as claimed in claim 1, wherein the control system communicates with the service request module and data transfer module via a network, such as a local or wide area network.

11. The system as claimed in claim 1, further comprising a user interface configured to display a plurality of analysis options, each being associated with a respective third party service module, and to allow a user to select one or more of the analysis options, wherein such selection leads to the generation of one or more service requests to provide analysis of the data from the support structures using third party application software contained in the respective third party service module(s).

12. The system as claimed in claim 1, wherein the third party application software contained on each of the third party service modules is configured, when run, to analyze sensor data from the respective monitoring device and output one or more features of a person that are determined from analyzing the sensor data.

13. The system as claimed in claim 1, wherein the output data corresponds to raw sensor data, or data processed from raw sensor data, that has been output from sensors located on or within the monitoring device.

14. The system as claimed in claim 1, wherein the monitoring device(s) are configured to analyse sensor data, and process the sensor data to determine one or more health features of the person, wherein the health features form at least some of the data required to execute the respective third party application software.

15. The system as claimed in claim 1, wherein the monitoring devices are configured to output movement data, wherein the movement data forms at least some of the data required to execute the respective third party application software.

16. The system as claimed in claim 1, wherein the control system is implemented on the same network as the service request module and/or data transfer module.

17. The system as claimed claim 1, in wherein the service request module is configured to determine if the data identified in the data request by the third party service module is appropriate, and approve the data request for further transmission to the control system if it is determined that the data identified in the data request is appropriate.

18. The system as claimed in claim 1, wherein the data transfer module is configured to verify that the data being transferred between the control system and the third party service module corresponds only to data identified in the data request, and forward the data from the control system to the third party service module only if this is the case.

19. The system as claimed in claim 1, wherein the third party service module is configured to send the analysis back to the control system via the data transfer module.

20. The system as claimed in claim 1, wherein the only data that is transferred between the control system and the third party are the service request, data request, data from the monitoring device and analysis.

21. The system as claimed in claim 1, wherein each of the monitoring devices is configured to output a variety of sensory data.

22. The system as claimed in claim 1, wherein the data transfer module is configured to log the amount and/or type of data transferred between the control system and the third party service module.

23. A method of exchanging data using a system as claimed in any preceding claim, the method comprising:
using the service request module to receive a service request and then communicate the service request to a respective one of the third party service modules;
using the respective third party service module to analyze the service request, determine the data required to provide the analysis of the data from the respective device using the third party application software, and then communicate a data request to the service request module;
using the service request module to receive the data request, and then to transmit this to the control system so as to notify the control system of the data required to provide the analysis of the data from the respective device using the third party application software;
setting up a data connection between the control system and the respective third party service module via the data transfer module; and
transferring the data required to provide the analysis from the control system to the third party service module, via the data transfer module.

24. The method as claimed in claim 23, further comprising the respective third party service module executing the third party application software so as to provide the analysis of the data.
